(19) United States
(12) Patent Application Publication
(10) Pub. No.: US 2003/0083562 A1
(43) Pub. Date: May 1, 2003

(54) Patient positioning system employing surface photogrammetry

(76) Inventors: Ali Bani-Hashemi, Walnut Creek, CA (US); Michelle Marie Svatos, Oakland, CA (US)

Correspondence Address: Siemens Corporation, Attn: Elsa Keller, Legal Administrator, Intellectual Property Department, 186 Wood Avenue South, Iselin, NJ, 08830, US

(21) Appl. No.: 10/004,363

(22) Filed: Nov. 1, 2001

(51) Int. Cl. A61B 5/05

(52) U.S. Cl. 600/407

(57) ABSTRACT

A system includes acquisition of first data representing a three-dimensional surface of at least a portion of a patient's body while the patient is in a first position, and acquisition of second data representing at least one internal portion of the patient's body while the patient is in the first position. The acquired data may be used to ensure correct patient positioning during treatment, to verify patient identity prior to treatment, to identify a change in a patient's body that may require revision of an associated treatment plan, and in conjunction with data representing a physical layout of a radiation treatment area to generate a radiation treatment plan.

### CROSS-REFERENCE TO RELATED APPLICATIONS

This application is related to commonly owned U.S. patent application Ser. No. ______, filed ______ (on even date herewith), Attorney Docket No. 2001 P 19664 US for “PATIENT POSITIONING SYSTEM EMPLOYING SURFACE PHOTOGRAMMETRY AND PORTAL IMAGING”, the contents of which are incorporated by reference in their entirety for all purposes.

### BACKGROUND OF THE INVENTION

1. Field of the Invention

The present invention relates generally to radiation treatment, and more particularly to facilitating patient positioning during such treatment.

2. Description of the Related Art

Computed Tomography (CT) is a tool used to plan modern radiation therapy. Under direction of an oncologist, a CT device generates multiple X-ray images of a patient and assimilates the images into a two-dimensional cross-sectional CT image of the patient's body. Unlike traditional X-ray images, a CT image depicts both hard objects such as bone and soft tissue including tumors. As a result, the CT image may be used for diagnosis, to delineate diseased tissue and healthy organs-at-risk, to define a treatment isocenter, and to design properties of a radiation beam usable to treat the patient (e.g., beam type, shape, dosage, duration).

In order to create a CT image, the patient is carefully positioned so as to permit x-ray radiation emitted by the CT device to intercept only an area of the patient's body that is of interest, and to avoid tissue in other areas. Immobilization devices and radiation shields are often used to achieve these ends. Accordingly, CT images not only fail to represent many areas of the patient's body, but also often fail to show devices, shields, and other accessories used to avoid unnecessary delivery of radiation to the patient.

As described above, CT images may be used to determine a radiation treatment plan. The plan is designed by a physicist, dosimetrist and/or attending physician based on the CT images and on the known configuration and capabilities of a radiation treatment device. However, the absence of the above-described elements from CT images may result in the determination of an inappropriate or unexecutable treatment plan.

For example, Intensity Modulated Radiation Treatment (IMRT) and Conformal Radiation Treatment (CRT) are popular types of treatments that are believed to maximize the treatment of tumors while minimizing the exposure of healthy tissue to harmful rays. Each of these treatments often requires the placement of a gantry and/or table of a radiation treatment device at various positions relative to one another in order radiate tumors from multiple directions. However, using current CT images, it is difficult to determine whether positioning the patient's body as dictated by the table positions and using immobilization devices and/or radiation shields will cause the body, devices or shields to collide or otherwise interfere with the gantry or other elements of the radiation treatment device. This difficulty is primarily caused by the failure of the CT images to include all of the physical elements described above.

Due to the foregoing, treatment plans are often designed conservatively to allow for possible physical interference among the relevant elements, even at the expense of using an optimal treatment configuration. Alternatively, a treatment plan may be designed without regard to possible physical interference. In either case, a “dry run” of the treatment with a patient positioned for delivery may reveal that the treatment is not feasible. This revelation requires design of a new treatment plan, which may result in a significant loss of time and money. Accordingly, the foregoing problems decrease the effectiveness of radiation treatment while also increasing its costs.

What is therefore needed is a system and method that would capture physical elements not captured by CT images. Further advantages would result from a system and method in which the captured elements could be used to assist in determining a radiation treatment plan.

Turning to the radiation treatment itself, conventional radiation treatment typically involves directing a radiation beam at a tumor in a patient to deliver a predetermined dose of therapeutic radiation to the tumor according to an established treatment plan. A suitable radiation therapy device is described in U.S. Pat. No. 5,668,847 issued Sep. 16, 1997 to Hernandez, the contents of which are incorporated herein for all purposes.

Healthy tissue and organs are often in the treatment path of the radiation beam during radiation treatment. The healthy tissue and organs must be taken into account when delivering a dose of radiation to the tumor, thereby complicating determination of the treatment plan. Specifically, the plan must strike a balance between the need to minimize damage to healthy tissue and organs and the need to ensure that the tumor receives an adequately high dose of radiation. In this regard, cure rates for many tumors are a sensitive function of the radiation dose they receive.

Treatment plans are therefore designed to maximize radiation delivered to a target while minimizing radiation delivered to healthy tissue. However, a treatment plan is designed assuming that relevant portions of a patient will be in a particular position during treatment. If the relevant portions are not positioned exactly as required by the treatment plan, the goals of maximizing target radiation and minimizing healthy tissue radiation may not be achieved. More specifically, errors in positioning the patient can cause the delivery of low radiation doses to tumors and high radiation doses to sensitive healthy tissue. The potential for misdelivery increases with increased positioning errors.

Due to the foregoing, treatment plans are designed under the assumption that positioning errors may occur that may result in misdelivery of radiation. Treatment plans compensate for this potential misdelivery by specifying lower doses or smaller beam shapes (e.g., beams that do not radiate edges of a tumor) than would be specified if misdelivery was not expected. Such compensation may decrease as margins of error in patient positioning decrease.

When used in conjunction with conventionally-designed treatments, more accurate positioning reduces the chance of harming healthy tissue. More accurate patient positioning also allows the design of more aggressive treatments. Specifically, if a margin of error in patient positioning is known to be small, treatment may be designed to safely radiate a greater portion of a tumor with higher doses than in cases where the margin of error is larger.

Accuracy in delivering radiation to a tumor decreases as a patient's body changes. For example, a treatment plan may specify that a particular radiation beam be delivered to a patient while the patient is in a particular position adjacent to a radiation treatment device. The beam may be successful in properly radiating a growth within the patient during initial treatments. However, the patient's body changes as time passes due to weight loss or other radiation symptoms. Eventually, the beam will not properly radiate the growth even if the patient is placed at the particular position prescribed by the treatment plan, because the growth is no longer at a same position relative to the treatment device as it was during the initial treatments.

### SUMMARY OF THE INVENTION

Some embodiments of the present invention provide a system, method, apparatus, and means to acquire first data representing a three-dimensional surface of at least a portion of a patient's body while the patient is in a first position, and to acquire second data representing at least one internal portion of the patient's body while the patient is in the first position. In further embodiments, a radiation treatment plan is determined based on the first data, the second data, and on data representing a physical layout of a radiation treatment station.

In other embodiments, the first position is a position that is substantially maintained during a computed tomography scan, and third data representing a three-dimensional surface of at least a portion of the patient's body is acquired while the patient is in a second position substantially maintained in preparation for radiation treatment. Further, in some embodiments it is determined, based on the first data and the third data, that the second position does not correspond to the first position.

According to some embodiments, it is determined, based on the first data and the third data, that the patient's body has changed by greater than a threshold amount, and, in response to the determination that the patient's body has changed by greater than the threshold amount, fourth data representing a three-dimensional surface of at least a portion of the patient's body is acquired while the patient is in a third position substantially maintained during a second computed tomography scan.

In other embodiments, third data representing a three-dimensional surface of at least a portion of the patient's body is acquired while the patient is in a second position, and a radiation beam is activated according to a radiation treatment plan if it is determined based on the third data that the second position corresponds to a point in a cycle of body motion specified by the treatment plan. Further, fourth data representing a three-dimensional surface of at least a portion of the patient's body may be acquired while the patient is in a third position, and the radiation beam may be deactivated according to a radiation treatment plan if it is determined based on the fourth data that the third position does not correspond to the point specified by the treatment plan.

The present invention is not limited to the disclosed preferred embodiments, however, as those skilled in the art can readily adapt the teachings of the present invention to create other embodiments and applications.

### BRIEF DESCRIPTION OF THE DRAWINGS

The exact nature of this invention, as well as its objects and advantages, will become readily apparent from consideration of the following specification as illustrated in the accompanying drawings, in which like reference numerals designate like parts, and wherein:

FIG. 1 is diagram illustrating a CT room according to some embodiments of the present invention;

FIG. 2 is a block diagram illustrating elements of devices according to some embodiments of the present invention;

FIG. 3 is a diagram illustrating a radiation treatment room according to some embodiments of the present invention;

FIG. 4 is a diagram illustrating elements of devices according to some embodiments of the present invention;

FIGS. 5a through 5d are flow diagrams illustrating process steps for using surface photogrammetry according to some embodiments of the present invention; and

FIG. 6 is a view of a phantom used to calibrate a system according to embodiments of the present invention.

### DETAILED DESCRIPTION

The following description is provided to enable any person skilled in the art to make and use the invention and sets forth the best modes contemplated by the inventors for carrying out the invention. Various modifications, however, will remain readily apparent to those in the art.

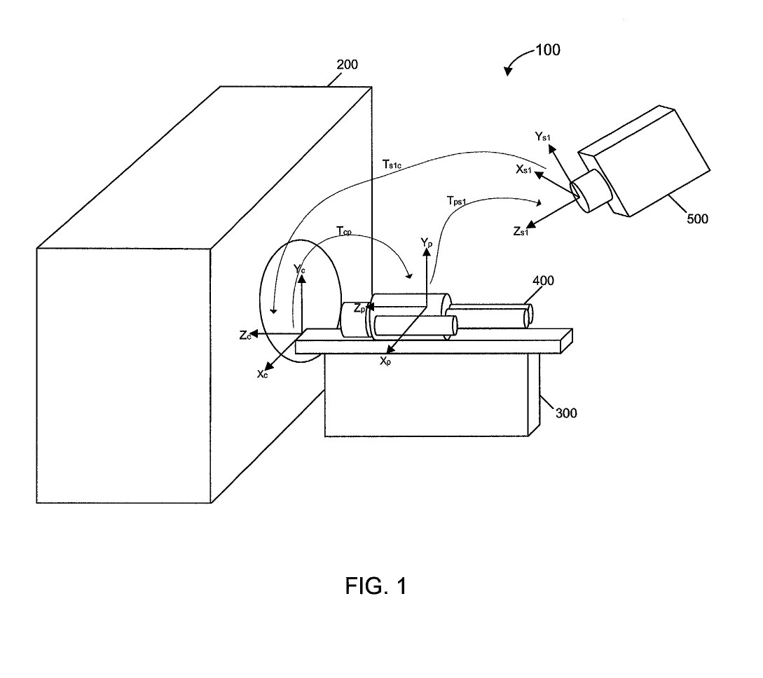

Turning now to the drawings, FIG. 1 illustrates CT room 100 configured to acquire data in accordance with some embodiments of the present invention. CT room 100 includes CT device 200, CT table 300, patient 400, and surface imager 500. The coordinate axes shown in FIG. 1 and the arrows connecting the axes will be described below and will therefore be ignored in the present discussion of the elements of FIG. 1.

CT device 200 is used to obtain CT data representing at least a portion of patient 400. Specifically, CT device acquires CT data by exploiting the x-ray principal: as x-rays pass through the body they are absorbed or attenuated at differing levels, thereby creating a matrix or profile of x-ray beams of different strength. In conventional x-ray imaging, an image of the profile is produced using film that is sensitive to x-rays. In the case of CT, the film is replaced by a banana-shaped detector that measures the x-ray profile and outputs data representing the profile.

The detector is mounted on a rotating frame inside CT device 200. Mounted opposite to the detector is an x-ray tube that emits a fan beam of x-rays as the rotating frame spins the x-ray tube and detector around patient 400. As the x-ray tube and detector spin, the detector measures profiles of the attenuated x-ray beam. Typically, in one 360° spin, about 1,000 profiles are measured. Each profile is subdivided spatially by the detector and fed into about 700 individual data channels. Each profile is then reconstructed into a two-dimensional image of the portion or “slice” that was scanned. The two-dimensional images may be processed to create a three-dimensional image. Both the two-dimensional images and the three-dimensional image are referred to herein as CT data, and both show tissue as well as bone. In some embodiments, the acquired CT data is represented in a CT coordinate frame, depicted by axes x_{c}, y_{c}, and z_{c} of FIG. 1.

CT table 300 is used to position a patient before, during and after acquisition of CT data. As such, CT table 300 is capable of moving so as to place relevant portions of the patient 400 in the path of the x-ray beam within CT device 200. This movement may be under the control of an operator and/or a computer program. It should be noted that any currently or hereafter-known CT table and CT device may be used in accordance with the present invention.

Surface imager 500 acquires a range image representing a three-dimensional surface within CT room 100. A range image is a picture in which each pixel value encodes not the intensity of light reflected in a certain direction but rather the distance (or range) of the nearest surface in that direction. The surface may include surfaces of patient 400, table 300, CT device 400, positioning accessories used to position patient 400, and shields used to protect portions of patient 400. Surface imager 500 may acquire the data of the range image using any suitable technique, such as stereo video acquisition or time-of-flight laser detection. In the present description, surface imager 500 acquires three-dimensional surface data by projecting a light pattern onto a surface and by sensing how the light pattern coats the surface. Of course, data acquired by surface imager 500 need not be in a range data format; any format usable to represent three-dimensional surface data will suffice.

In some embodiments, the elements of room 100 operate to acquire first data representing a three-dimensional surface of at least a portion of a patient's body while the patient is in a first position, and to acquire second data representing at least one internal portion of the patient's body while the patient is in the first position. These features advantageously allow treatment planners to efficiently visualize relationships between CT device 200, CT table 300 and properly-positioned patient 400 for a variety of treatment scenarios.

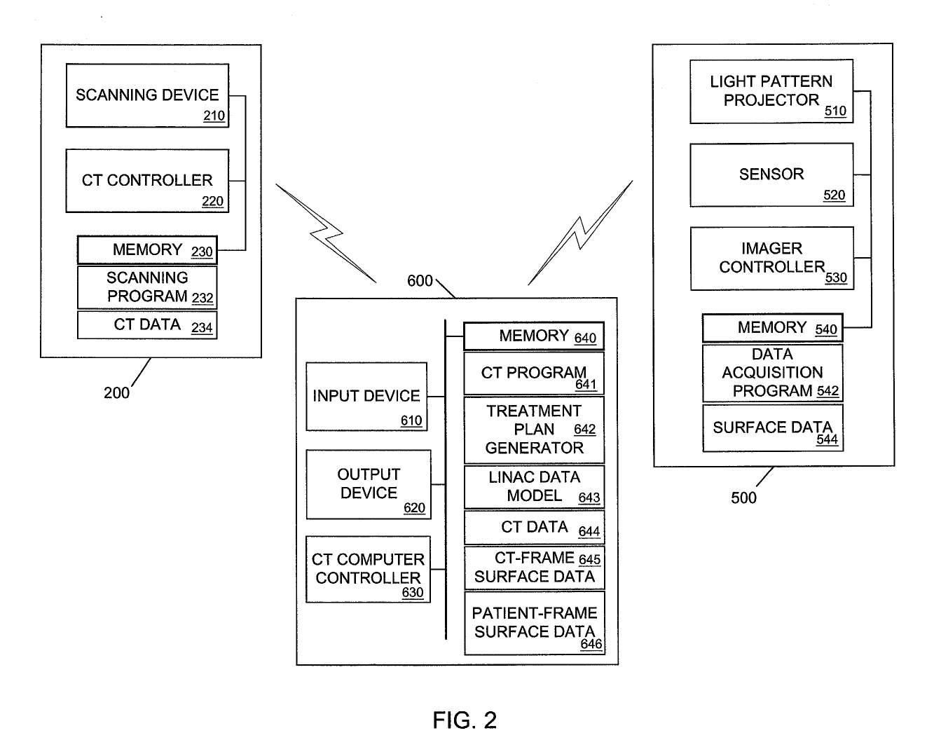

FIG. 2 illustrates internal architectures of various elements of CT room 100, including CT device 200 and surface imager 500. Also illustrated is an internal architecture of CT computer 600, which is not shown in CT room 100. CT computer 600 may be operated so as to cause CT device 200 to perform steps in accordance with embodiments of the present invention. CT computer 600 may be located within CT room 100, in a radiation-proof room adjacent to CT room 100, or elsewhere.

As shown, CT device 200 includes scanning device 210, which includes the x-ray tube and detector described above as well as other physical devices needed to generate x-ray profiles. CT controller 220 controls scanning device 210 using internal logic and/or executable process steps. Accordingly, scanning device 210 may comprise a microprocessor, a programmable logic controller or the like. Some of these process steps may be part of scanning program 232 stored in memory 230. In this regard, scanning program 232 includes executable process steps for controlling the hardware elements of CT device 100 to scan a body and to thereby generate x-ray profiles. The generated x-ray profiles are stored in memory 230 as CT data 234. CT data 234 may include raw profile data, two-dimensional images generated based on raw profile data, and three-dimensional images generated based on raw profile data and/or two-dimensional images.

CT computer 600 includes input device 610, output device 620, CT computer controller 630, and memory 640. Input device 610 may be manipulated by an operator to submit commands to CT computer 600 and to CT device 200. Input device 610 may therefore comprise one or more of a keyboard, a pointing device, a touch screen or any other input device. Output device 630 is used to output images, data and text to the operator, and therefore may comprise a display, a printer, and the like. Data may also be input to and output from CT computer 600 using a communication port (not shown) that links CT computer 600 to other devices. For example, commands may be transmitted to and CT data may be received from CT device 200 over such a communication port.

CT computer controller 630 controls elements of CT computer 600 according to internal logic and/or executable process steps. The process steps may be received from another device or stored in memory 640. Process steps used to control the functions of CT device 200 are found in CT program 641. Treatment plan generator stores process steps that are executable to generate a radiation treatment plan based on CT data, surface data, and data of Linac (Linear Accelerator) data model 643.

In this regard, Linac data model 643 includes data representing a physical structure of elements within a Linac room in which radiation treatment is to be delivered. The data may be usable by any conventional computer application for generating treatment plans. By generating a radiation treatment plan based on the surface data and the data model, potential interference between a patient's body and the elements of the Linac room can be accurately determined. As a result, the treatment plan is less likely to require costly revision and may be more aggressive than otherwise.

Also stored in memory 640 are CT data 644, CT-frame surface data 645 and patient-frame surface data 646. CT data 644 merely includes CT data generated by CT device 200 in any format, including raw and/or image format. In some embodiments, the data of CT data 644 is represented in the coordinate frame of CT device 200. CT-frame surface data 645 includes three-dimensional surface data generated by surface imager 500 that has been transformed to the coordinate frame of CT device 200. Patient-frame surface data 646 also includes three-dimensional surface data generated by surface imager 500, but the surface data of surface data 646 has been transformed to the coordinate frame of a particular patient. In this regard, surface data 646 may include individual sets of surface data each corresponding to a different patient. Thorough descriptions of the above-mentioned transformations are set forth below.

As shown in FIG. 2, surface imager 500 includes light pattern projector 510, sensor 520, image controller 530 and memory 540. Light pattern projector 510 and sensor 520 are controlled by image controller 530 to acquire range data representing a three-dimensional surface as described above. Image controller 530 may exert this control by executing process steps of data acquisition program 542. The acquired surface data is also stored in memory 540 as surface data 544. Surface data 544 may include several sets of surface data representing portions of different patient's bodies. In some embodiments, surface data 544 includes range data that has been transformed to the coordinate frame of CT device 200.

Of course, each of the devices shown in FIG. 2 may include less or more elements than those shown. Moreover, transformation and storage of acquired data may be performed by any one or more of the devices. In addition, embodiments of the invention are not limited to the three devices shown.

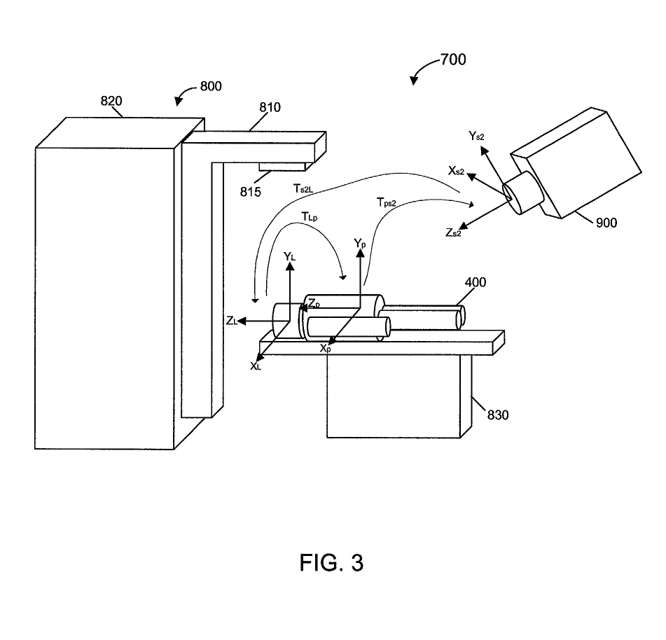

FIG. 3 illustrates Linac room 700 according to some embodiments of the invention. Linac room 700 includes patient 400, Linac 800 and surface imager 900. As mentioned with respect to FIG. 1, descriptions of the illustrated coordinate axes and connecting arrows will be postponed until later in the present specification.

As shown, Linac 800 includes gantry 810, base 820 and Linac table 830. Gantry 810 contains treatment head 815 from which a beam of radiation is emitted. The beam may comprise electron, photon or any other type of detectable radiation. Gantry 810 can be swiveled around a horizontal axis of rotation during radiation treatment so as to allow different beam angles and radiation distributions without having to move the patient 400.

During a course of treatment, the radiation beam is trained on the Linac isocenter, located at the intersection of axes X_{L}, Y_{L} and Z_{L}. Accordingly, patient 400 is preferably positioned so that the center of an area to be radiated, or the patient isocenter (located at the intersection of axes Xₚ, Yₚ and Zₚ), is located at the Linac isocenter. Therefore, the position of patient 400 in Linac room 700 is not optimal for delivering treatment. More specifically, patient 400 will be positioned prior to treatment so that the patient isocenter and the Linac isocenter coincide.

Surface imager 900 is used to acquire surface data representing a three-dimensional surface within Linac room 700. The data, which may comprise range data, may be used to position patient 400 for delivery of treatment. More specifically, the acquired surface data may be used in conjunction with surface data acquired by surface imager 500 during a CT scan to substantially duplicate, on Linac table 830, a position of at least a portion of a patient's body that was maintained during the CT scan. Surface imager 900 may be identical to surface imager 500, may be a different model of surface imager that utilizes a same operational principle as imager 500, or may be a surface imager operating in an entirely different manner from imager 500.

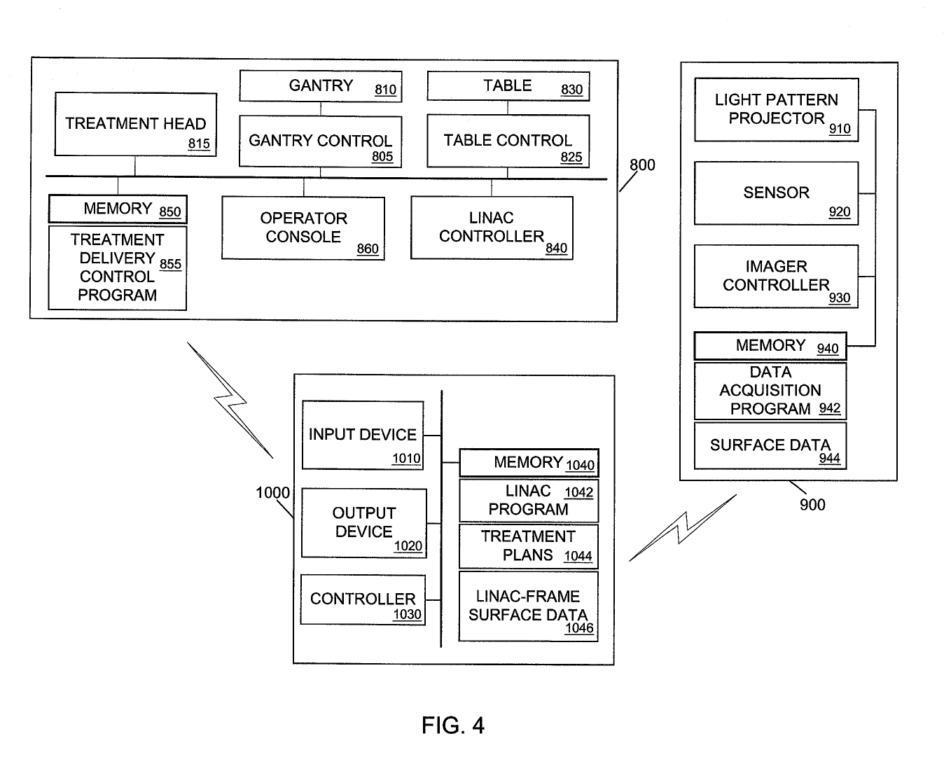

Referring now to FIG. 4, a block diagram is shown depicting portions of Linac 800, surface imager 900 and Linac computer 1000. Linac computer 1000 is not shown in FIG. 3 because Linac computer 1000 is typically operated by a therapist who is located in a different room so as to be protected from radiation. The therapist administers actual delivery of radiation treatment plan generated based on, in some embodiments, CT data representing at least one internal portion of a patient's body, surface data representing a three-dimensional surface of the patient as positioned for the CT scan, and data representing a physical layout of Linac room 700.

The therapist operates Linac computer 1000 by using input device 1010, such as a keyboard or the like. Data can be input from other devices such as CT computer 600 via an I/O port (not shown). Various data can be output to the therapist before and during treatment via output device 1020.

Memory 1040 stores data for controlling and generated by Linac 800. This data includes process steps of Linac program 1042 which are executed by controller 1030 to provide control over Linac 800 so as to execute one of treatment plans 1044 defined by an oncologist for a particular patient. One or more of treatment plans 1044 may be generated by CT computer 600 using treatment plan generator 642 and transmitted to Linac computer 1000 via any type of communication link usable to transmit data. Of course, treatment plans 1044 may be generated by Linac computer 1000 using Linac program 1042. In this regard, the functions described herein as being performed by one of CT computer 600 and Linac computer 1000 may be performed by a single device or by other devices including CT device 200, surface imager 500, Linac 800 and surface imager 900. Those skilled in the art will also appreciate that any suitable general purpose or specially programmed computer may be used to achieve the functionality described herein.

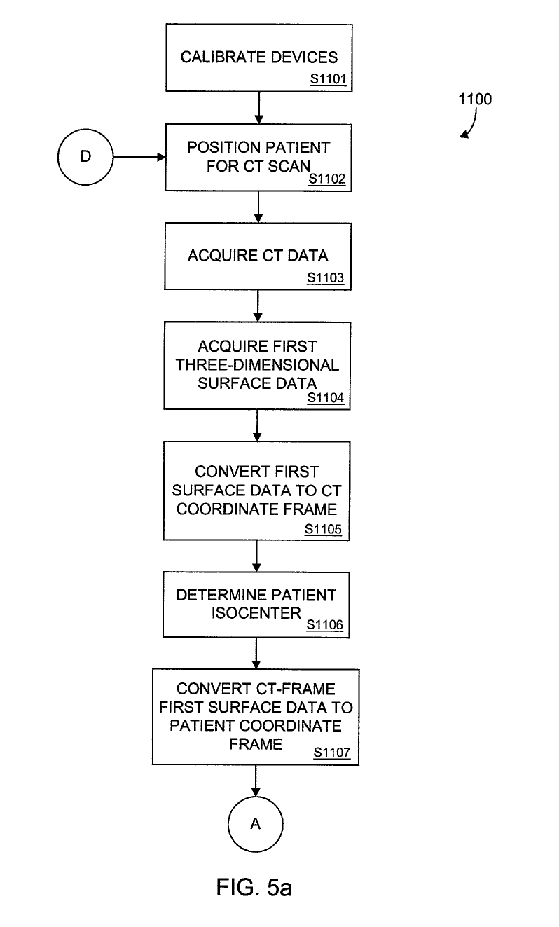

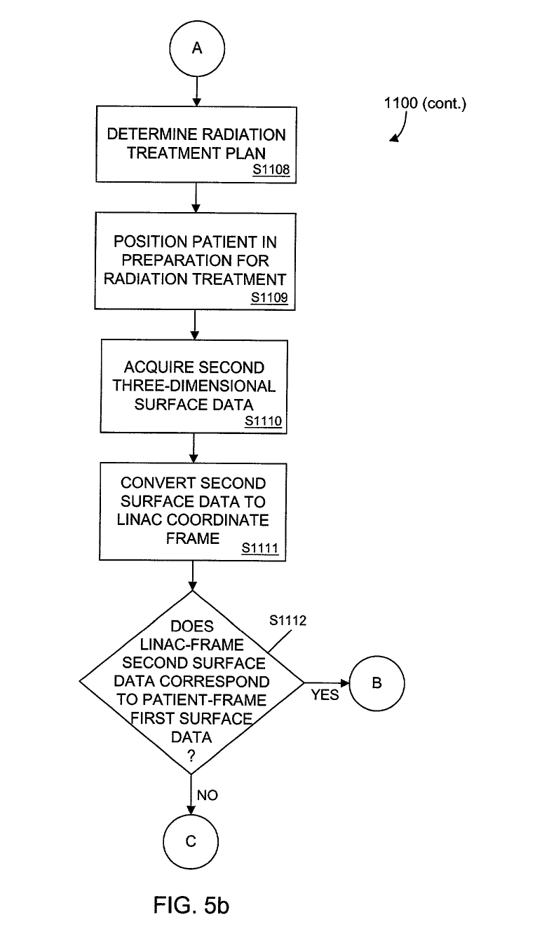

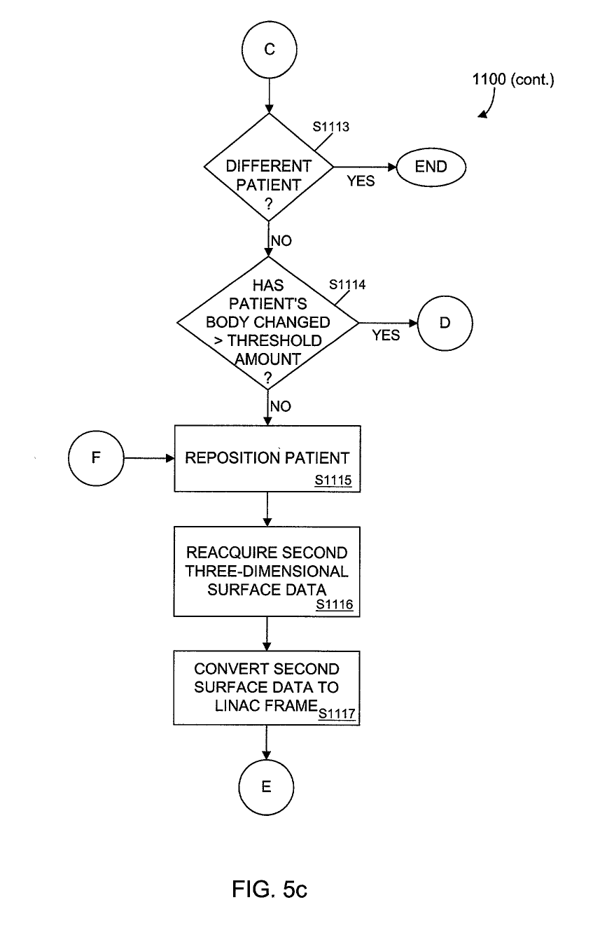

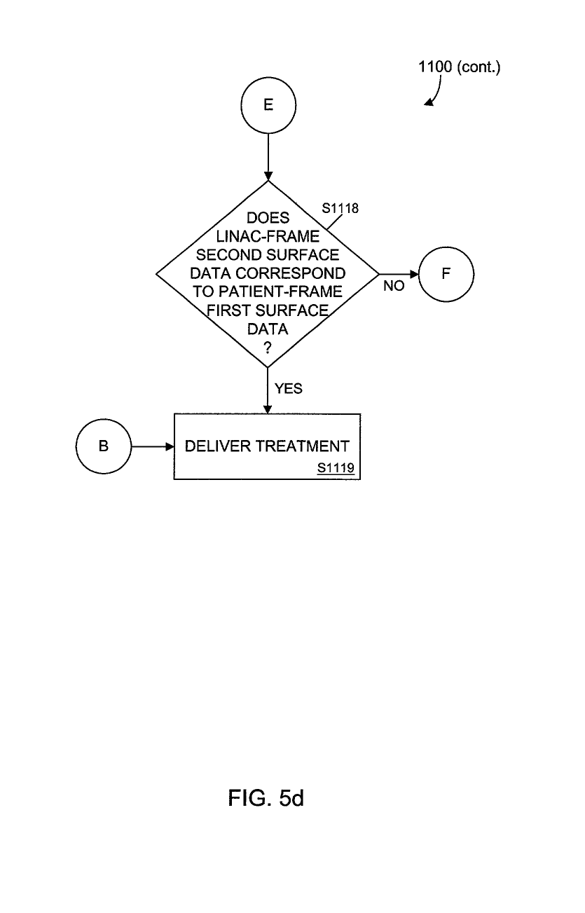

Linac-frame surface data 1046 is also stored in memory 1040. Linac-frame surface data 1046 is used to determine if a patient is correctly positioned according to a radiation treatment plan. Details of this process are set forth below with respect to FIGS. 5a through 5d. According to some embodiments, controller 1030 executes process steps of Linac program 1042 to convert surface data generated by surface imager 900 to Linac-frame surface data 1046. In this regard, surface imager 900 in the present example is identical to surface imager 500 and a discussion of its physical elements will therefore be omitted. In operation, however, surface imager 900 acquires data representing a three-dimensional surface of at least a portion of a patient's body while the patient is in a position substantially maintained in preparation for radiation treatment. This data, stored among surface data 944, is used to determine whether the position corresponds to a position maintained by the patient during acquisition of CT data that is used to plan the radiation treatment.

Radiation treatment is delivered by treatment head 815 under control of Linac controller 840. Particularly, Linac controller 840 executes process steps of treatment delivery control program 855 to generate and deliver a beam of radiation according to a treatment plan such as those stored among treatment plans 1044. In this regard, Linac computer 1000 may transmit instructions according to a treatment plan to Linac 800, which in turn executes those instructions using functions provided by treatment delivery control program 855.

For example, some of the instructions may require Linac controller 840 to issue a command to gantry control 805 to rotate gantry 810 to a specified position relative to patient 400. Other instructions may require table control 825 to move table 830 to an appropriate position so as to position patient 400 properly with respect to treatment head 815. In some embodiments, gantry 810 and/or table 830 may be repositioned during a treatment to deliver a prescribed dose of radiation. Many functions of Linac 800 may also be controlled by an operator manually using operator console 860, which may a hard or wireless-linked remote control device.

FIGS. 5a through 5d illustrate process steps 1100 according to some embodiments of the present invention. Process steps 1100 may be performed by various devices under the control of controller-executable process steps stored locally to the devices or received from other devices. The following description of process steps 1100 associates each process step with a device that performs the step, and also mentions two or more alternative devices for performing some process steps. Of course, embodiments of the present invention may differ from the description. The particular arrangement of process steps 1100 are not meant to imply a fixed order to the steps; embodiments of the present invention can be practiced in any order that is practicable.

Briefly, process steps 1100 execute to acquire first data representing a three-dimensional surface of at least a portion of a patient's body while the patient is in a first position, and to acquire second data representing at least one internal portion of the patient's body while the patient is in the first position. Moreover, steps 1100 execute to determine a radiation treatment plan is determined based on the first data, the second data, and on data representing a physical layout of a radiation treatment station.

Initially, in step S1101, CT device 200 and surface imager 500 are calibrated. As shown in FIG. 1, CT device 200 acquires CT data that is represented in a coordinate frame illustrated by axes X_{c}, Y_{c} and Z_{c}. This coordinate frame will be referred to as the CT frame. Surface imager 500 acquires three-dimensional surface data formatted with respect to a coordinate frame illustrated by axes Xₛ₁, Yₛ₁ and Zₛ₁. This frame will be referred to as the first imager frame. Calibration consists of determining a transformation matrix T_{s1c} for converting data represented in the first imager frame to data represented in the CT frame.

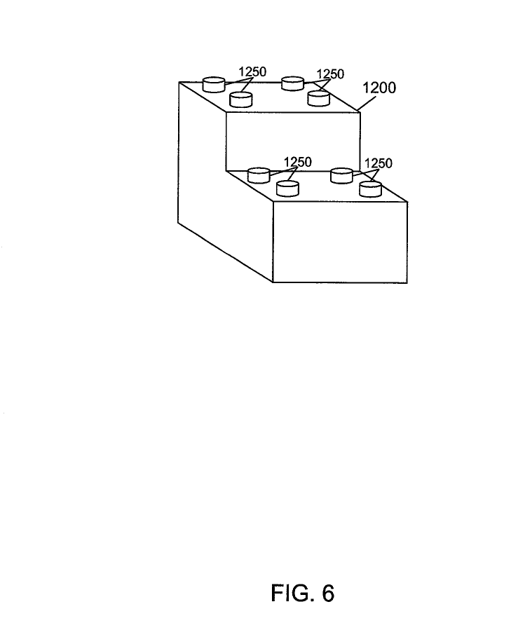

FIG. 6 illustrates phantom 1200 used to determine transformation matrix T_{s1c} according to some embodiments of step S1101. The body of phantom 1200 consists of a material with a low x-ray absorption coefficient, such as acrylic. Phantom 1200 includes eight fiducial markers 1250 that may be sensed by CT device 200 as well as by surface imager 500, and which possess an x-ray absorption coefficient that is relatively higher than the body's coefficient.

More specifically, phantom 1200 is placed at the intersection of axes X_{c}, Y_{c} and Z_{c} while CT table 300 is at the zero position shown in FIG. 1. Phantom 1200 is then scanned by CT device 200, thereby generating CT data represented in the CT frame. Table 300 is returned to the zero position and surface imager 500 acquires three-dimensional surface data representing phantom 1200. Because they extend from the body of phantom 1200, the acquired data will represent fiducial markers 1250. Coordinates of eight points representing markers 1250 are identified from each of the CT data and the surface data. The coordinates are used to generate an over-determined set of linear equations, the solution of which is T_{s1c} Preferably, phantom 1200 includes at least four non-coplanar corresponding points that may be used to solve for T_{s1c}, using known matrix techniques. T_{s1c} may be stored in memory 640 of CT computer 600. In this regard, step S1101 may be performed by CT device 200 and surface imager 500 under control of CT computer 600.

Step S1101 also includes calibration of Linac 800 and surface imager 900. This calibration is intended to produce transformation matrix T_{s2L}, which may be used to convert data acquired by surface imager 900 to a coordinate space of data acquired by Linac 800.

Linac table 830 is initially moved to its zero position as shown in FIG. 3. FIG. 3 also shows coordinate axes X_{L}, Y_{L} and Z_{L} representing a Linac coordinate frame and axes Xₛ₂, Yₛ₂ and Zₛ₂ representing a coordinate frame of surface imager 900, hereafter referred to as a second imager coordinate frame. Phantom 1200 is placed at the origin of the Linac coordinate frame and surface imager 900 acquires data representing a three-dimensional surface of phantom 1200. Coordinates of fiducial markers 1250 are extracted from the acquired data.

Next, Linac table 830 is moved so as to position one of markers 1250 at the isocenter of Linac 800. The isocenter is a point to which a radiation would be focused if Linac were activated. In FIG. 3, the isocenter lies at the origin of the Linac coordinate frame. The coordinates of Linac table 830 are recorded and table 830 is moved so as to position another of markers 1250 at the isocenter of Linac 800. Again the coordinates of table 830 are recorded. The above process is repeated for each of markers 1250. As described with respect to T_{s1c}, the eight coordinates acquired by surface imager 900 and the eight table coordinates are used to generate an over-determined set of linear equations, the solution of which is T_{s2L}.

Of course, the phantoms used to calibrate in CT room 100 and in Linac room 700 need not be identical. Moreover, embodiments of the invention may utilize methods of determining each of the transformation matrices that are different than that described above.

Flow continues from step S1101 to step S1102, in which a patient is positioned for a CT scan in CT room 100. The patient's body is positioned on CT table 300 in a manner intended to produce a best-quality CT data of a specific internal portion of the patient. As described in the Background, such positioning may require the creation and/or use of pillows, wedges, supports or shields. Once the patient is adequately positioned, CT device acquires CT data in step S1103 as described above. The acquired CT data is stored among CT data 234 and CT data 644, and is represented in the CT coordinate frame.

In step S1104, surface imager 500 executes data acquisition program 542 to acquire data representing a three-dimensional surface of the patient's body. The three-dimensional surface is intended to substantially mimic a surface of the patient's body and other physical elements as positioned during acquisition of the CT data. Accordingly, it may be beneficial to perform step S1104 contemporaneously with step S1103.

The surface data is stored among surface data 544 and is represented in the first imager coordinate frame. Accordingly, the surface data is converted to the CT coordinate frame in step S1105. In the present embodiment, the conversion is performed by CT computer 600, which executes CT program 641 to apply transformation matrix T_{s1c} to the surface data. The converted data is then stored among CT frame surface data 645.

Next, a patient isocenter is determined in step S1106. The isocenter is a point within the patient's body on which a radiation beam should be focused according to a treatment plan. Accordingly, a position of the isocenter is determined by a specialist who examines graphic representations of the CT data acquired in step S1103. The representations may be displayed by output device 620 and/or may be presented by output device 620 in hardcopy form. It should be noted that, according to this embodiment, steps S1103 through S1106 may be performed in any order, as long as step S1103 occurs prior to step S1106, and step S1104 occurs prior to step S1105.

It will be assumed that the patient isocenter is determined to be located at the intersection of axes Xₚ, Yₚ and Zₚ of FIG. 3. Using the coordinates of the isocenter with respect to the CT coordinate frame, the CT-frame surface data is converted in step S1107 to the coordinate frame defined by axes Xₚ, Yₚ and Zₚ, or the patient coordinate frame. The conversion may be performed by CT computer 600, and the converted data may be stored among patient-frame surface data 646.

A radiation treatment plan is determined in step S1108 based on the acquired CT data, the acquired surface data and on data representing a physical layout of a radiation treatment station. The latter data is included in Linac data model 643, and includes models of gantry 810, base 820, Linac table 830 and of any other element that may physically interfere with patient 400 during radiation treatment. The treatment plan may be determined by operating CT computer 600 to execute treatment plan generator 642.

In some embodiments, one or more specialists view superimposed representations of the CT data, the surface data and the physical layout data to determine how best to treat tissue located at the determined patient isocenter. In order to simplify processing required by CT computer 600 to superimpose the representations, the surface data may be represented in the CT coordinate frame. Of course, treatment plan generator may include executable process steps to generate such a scenario using surface data represented in the first imager frame. Issues taken into account during step S1108 include gantry position, table position, beam shape, etc. The determined treatment plan may be transmitted to Linac computer 1000 for storage among treatment plans 1044.

In step S1109, patient 400 is positioned on Linac table 830 in preparation for radiation treatment. In some embodiments, the patient is positioned so that laser beams emitted from devices mounted in Linac room 700 intercept tattoos or other markings placed on the patient in CT room 100. According to some of these embodiments, a patient's body is marked at three or more points orthogonal to the determined isocenter. To mark the patient thusly, the patient is positioned on CT table 300 and CT computer 600 uses coordinates of the determined isocenter to position beam-emitting devices (not shown) orthogonal to the isocenter. The patient is marked where the beams intercept the patient's body. In Linac room 700, beam-emitting devices are mounted such that their emitted beams would intersect at the isocenter of Linac 800 if the beams intercepted the tattoos. Other conventional techniques may be used to position patient 400 in step S1109.

Surface imager 900 acquires data representing a three-dimensional surface of at least a portion of the patient's body in step S1110. The acquired data is represented in the second imager coordinate frame and stored among surface data 544. Next, in step S111, Linac computer 1000 converts the data acquired in step S1110 to the Linac coordinate frame using transformation matrix T_{s2L}. The converted data is stored among Linac-frame surface data 1046 of memory 1040.

In step S1112, Linac computer 1000 executes Linac program 1042 to determine if the surface data produced in step S111 corresponds to the surface data produced in step S1107. The data may be determined to correspond if the coordinates reflected in the data are identical or vary by less than a specified statistical, mathematical or distance threshold. The determination may only take into account surface data reflecting portions of patient 400 that lie within a certain distance of the Linac isocenter, and may include manual as well as automated steps. Since the surface data produced in step S1111 is represented in the Linac coordinate frame and the surface data produced in step S1107 is represented in the patient coordinate frame, determination of a correspondence in step S1112 indicates that the patient isocenter is located substantially at the Linac isocenter and that a relevant surface of patient 400 is substantially at the same position as it was in step S1104. Accordingly, flow proceeds to step S119 for delivery of radiation treatment according to the radiation treatment plan determined in step S1108.

If the data are determined not to correspond, then the patient isocenter is not located substantially at the Linac isocenter, a relevant surface of patient 400 is not substantially at the same position as it was in step S1104, or both. Flow therefore continues to step S1113, wherein it is determined if the patient positioned in step S1109 is the same patient positioned in step S1102. This determination advantageously may prevent delivery to one patient of a radiation treatment plan designed for another patient. If the sets of data compared in step S1112 differ in any manner that indicates that the sets represent different patients, process steps 1100 terminate. The determination of step S1113 may include manual viewing of two superimposed surfaces represented by the two sets of data, automated analysis of the data sets, or any other process. If it is determined that the patient in Linac room 700 is not different from the patient from whom CT data was acquired in step S1109, flow continues to step S1114.

It is determined, in step S1114, if the patient's body has changed by an amount greater than a threshold amount. Over the course of radiation treatment, a patient often loses weight and therefore experiences changes to his physical structure. However, since a treatment plan is determined for a patient based on the patient's physical structure, the treatment plan may cease to be appropriate for the patient if the structure changes. Therefore, in a case that it is determined, based on the surface data produced in step S1111 and on the surface data produced in step S1107, that the patient's body has changed by an amount greater than a threshold amount, flow returns to step S1102 and continues therefrom in order to generate a new treatment plan in view of the body changes. Such features provide more accurate and effective treatment that previously available.

The data comparisons of steps S1112, S1113 and S1114 will be simplified if the patient is positioned in step S1109 so that the patient isocenter is located substantially at the Linac isocenter. In such a case, the sets of data may be directly compared since the data are represented in substantially identical coordinate frames. Of course, conventional data analysis techniques may be used to register the two sets of data in a same coordinate frame prior to comparing the data.

If the determination in step S1114 is negative, patient 400 is repositioned in step S1115. Repositioning in step S1115 may include any method of changing a position of patient relative to Linac treatment head 815, including one or more of instructing patient 400 to move, physically moving patient 400, rotating gantry 810, and moving Linac table 830. Patient 400 may be repositioned automatically by Linac controller 800 or Linac computer 1000 based on analyzed differences between the Linac-frame surface data and the patient-frame surface data, and/or manually by an operator using operator console 860 or input device 1010. The operator may be guided by instructions determined based on the analyzed differences and presented through console 860 or output device 1020. In some embodiments, the operator is presented with an image representing the patient-frame surface data superimposed on an image representing the Linac-frame surface data. As the patient is repositioned, the Linac-frame data is periodically re-acquired and the superimposed image representing the surface of patient 400 in Linac room 700 is periodically updated based on the re-acquired data.

Surface imager 900 acquires second data representing a three-dimensional surface of a portion of the body of patient 400 in step S1116. The second data is converted to the Linac coordinate frame in step S1117 in the manner described above with respect to step S1111. Then, in step S1118, it is determined whether the converted second surface data corresponds to the patient-frame surface data generated in step S1107. This determination may be performed using any of the techniques discussed with respect to step S1112. If the determination is negative, flow returns to step S115 and continues therefrom. If it is determined that the converted second surface data corresponds to the patient-frame surface data, the determined radiation treatment plan is delivered to patient 400 in step S1119.

For example, in an embodiment where electron radiation will be used to treat a patient, Linac computer 1000 may direct Linac 800 to deliver a particular dosage to the patient isocenter (which is substantially identical to the Linac isocenter after performance of process steps 100). In response, Linac controller 840 executes process steps of treatment delivery control program 855 that control beam delivery unit 840 to deliver the dosage. Such control may include positioning electron collimator leaves (not shown) so as to create a desired beam shape.

Those in the art will appreciate that various adaptations and modifications of the above-described embodiments can be configured without departing from the scope and spirit of the invention. For example, after an affirmative determination in step S1114, the treatment plan may be altered as an alternative to repositioning patient 400. In other words, the treatment plan may be modified to take into account differences between the patient-frame surface data acquired in CT room 100 and the Linac-frame surface data acquired in Linac room 700. After modifying the plan, the treatment plan may be immediately delivered.

In some embodiments, features of process steps 1100 may be used to provide gated radiation treatment. Gating involves the delivery of a specified radiation beam only when the patient's body is at a particular position corresponding to a point in a cycle of motion. For example, one gating treatment calls for delivery of a radiation beam at a point after exhalation and just prior to inhalation. To provide such treatment, the patient may be positioned according to process steps 100 and Linac-frame surface data is acquired and analyzed to determine if the position of the patient corresponds to the point. Once the determination is made, an appropriate radiation beam is delivered. Linac-frame surface data continues to be acquired and analyzed to determine whether the patient has moved to a position no longer corresponding to a point in the cycle of motion. Once this occurs, the beam is deactivated.

Those in the art will recognize that a number of portal imaging techniques may be utilized in conjunction with embodiments of the present invention to position a patient. Portal images are images of a patient portal through which a radiation beam passes. These images show internal bony structures of the patient as well as any implanted fiducials. Accordingly, portal images can be taken before or after treatment to ascertain that a patient position, as well as a beam shape, conforms to a desired treatment plan.

Moreover, it should be noted that functions ascribed to one device herein may be performed by other devices. In one example, the functions ascribed to CT computer 600 and to Linac computer 1000 are performed by a single computing device. In other examples, elements or functions described with respect to one of these devices are present or performed by the other.

What is claimed is:

1. A method comprising:
acquiring first data representing a three-dimensional surface of at least a portion of a patient's body while the patient is in a first position; and
acquiring second data representing at least one internal portion of the patient's body while the patient is in the first position.

2. A method according to claim 1, further comprising:
determining a radiation treatment plan based on the first data, the second data, and on data representing a physical layout of a radiation treatment station.

3. A method according to claim 2, wherein the step of determining the radiation treatment plan comprises:
determining a position of a radiation treatment device that will avoid the patient's body and that will allow irradiation of a portion of the at least one internal portion.

4. A method according to claim 1, wherein the first position is a position that is substantially maintained during a computed tomography scan, the method further comprising:
acquiring third data representing a three-dimensional surface of at least a portion of the patient's body while the patient is in a second position substantially maintained in preparation for radiation treatment.

5. A method according to claim 4, further comprising:
determining, based on the first data and the third data, that the second position does not correspond to the first position.

6. A method according to claim 5, further comprising:
instructing the patient to move so that the second position corresponds to the first position.

7. A method according to claim 5, further comprising:
changing a radiation treatment plan for the patient based on a difference between the first position and the second position.

8. A method according to claim 1, further comprising:
determining, based on the first data and the third data, that the patient represented by the first data is different from the patient represented by the third data.

9. A method according to claim 4, further comprising:
determining, based on the first data and the third data, that the patient's body has changed by greater than a threshold amount; and
in response to the determination that the patient's body has changed by greater than the threshold amount, acquiring fourth data representing a three-dimensional surface of at least a portion of the patient's body while the patient is in a third position substantially maintained during a second computed tomography scan.

10. A method according to claim 1, further comprising:
acquiring third data representing a three-dimensional surface of at least a portion of the patient's body while the patient is in a second position; and
activating a radiation beam according to a radiation treatment plan if it is determined based on the third data that the second position corresponds to a point in a cycle of body motion specified by the treatment plan.

11. A method according to claim 10, further comprising:
acquiring fourth data representing a three-dimensional surface of at least a portion of the patient's body while the patient is in a third position; and
deactivating the radiation beam according to a radiation treatment plan if it is determined based on the fourth data that the third position does not correspond to the point specified by the treatment plan.

12. A method comprising:
acquiring computed tomography data of a patient while the patient remains substantially in a first position;
acquiring first three-dimensional data representing a surface of the patient while the patient remains substantially in the first position;
determining a radiation treatment plan based on the computed tomography data, the three-dimensional data, and data representing a physical layout of a radiation treatment station;
acquiring second three-dimensional data representing a surface of the patient while the patient remains substantially in a second position at the radiation treatment station;
determining if the second three-dimensional data corresponds to the first three-dimensional data; and
delivering radiation to the patient according to the radiation treatment plan if it is determined that the second three-dimensional data corresponds to the first three-dimensional data.

13. A system comprising:
a computed tomography scanning device for acquiring computed tomography data of a patient while the patient is in a scanning position; and
a first surface photogrammetry device for acquiring first three-dimensional surface data of at least a portion of the patient's body while the patient is in the scanning position.

14. A system according to claim 13, further comprising:
a treatment planning device for generating a radiation treatment plan based on the computed tomography data, the first three-dimensional surface data, and data representing a physical layout of a radiation treatment station.

15. A system according to claim 13, further comprising:
a radiation treatment device for delivering radiation to the patient;
a second surface photogrammetry device for acquiring second three-dimensional surface data of at least a portion of the patient's body while the patient is in a treatment position on the radiation treatment device;
a controller for determining if the treatment position corresponds to the scanning position based on the first three-dimensional surface data and the second three-dimensional surface data.

16. A system according to claim 15, wherein the first surface photogrammetry device and the second surface photogrammetry device are a same device.

17. A medium storing controller-executable process steps, the process steps comprising:
a step to acquire first data representing a three-dimensional surface of at least a portion of a patient's body while the patient is in a first position; and
a step to acquire second data representing at least one internal portion of the patient's body while the patient is in the first position.

18. A medium according to claim 17, the process steps further comprising:
a step to determine a radiation treatment plan based on the first data, the second data, and data representing a physical layout of a radiation treatment station.

19. A medium according to claim 17, wherein the first position is a position that is substantially maintained during a computed tomography scan, the process steps further comprising:
a step to acquire third data representing a three-dimensional surface of at least a portion of the patient's body while the patient is in a second position substantially maintained in preparation for radiation treatment.

20. A medium according to claim 19, the process steps further comprising:
a step to determine, based on the first data and the third data, that the patient's body has changed by greater than a threshold amount; and
a step to acquire, in response to the determination that the patient's body has changed by greater than the threshold amount, fourth data representing a three-dimensional surface of at least a portion of the patient's body while the patient is in a third position substantially maintained during a second computed tomography scan.

21. A medium according to claim 17, the process steps further comprising:
a step to acquire third data representing a three-dimensional surface of at least a portion of the patient's body while the patient is in a second position; and
a step to activate a radiation beam according to a radiation treatment plan if it is determined, based on the third data, that the second position corresponds to a position specified by the treatment plan.

22. A medium according to claim 17, the process steps further comprising:
a step to acquire third data representing a three-dimensional surface of at least a portion of the patient's body while the patient is in a second position; and
a step to activate a radiation beam according to a radiation treatment plan if it is determined based on the third data that the second position corresponds to a point in a cycle of body motion specified by the treatment plan.

23. A medium according to claim 22, the process steps further comprising:
a step to acquire fourth data representing a three-dimensional surface of at least a portion of the patient's body while the patient is in a third position; and
a step to deactivate the radiation beam according to a radiation treatment plan if it is determined based on the fourth data that the third position does not correspond to the point specified by the treatment plan.
